(19) United States
(12) Patent Application Publication
(10) Pub. No.: US 2006/0271068 A1
(43) Pub. Date: Nov. 30, 2006

(54) Flexible tips for delivering materials and guiding fibers

(75) Inventor: Densen Cao, Sandy, UT (US)

Correspondence Address: Daniel P. McCarthy;Parsons Behle & Latimer, 201 South Main Street, Suite 1800, P.O. Box 45898, Salt Lake City, UT, 84111, US

(73) Assignee: Cao Group, Inc.

(21) Appl. No.: 11/411,194

(22) Filed: Apr. 25, 2006

Related U.S. Application Data
(60) Provisional application No. 60/674,597, filed on Apr. 25, 2005.

(51) Int. Cl. A61B 19/00

(52) U.S. Cl. 606/129; 606/108

(57) ABSTRACT

A flexible delivery tip for delivering flowable substances, fiber or wire. The delivery tip uses 2 or more snap-together plastic pieces that articulate with respect to each other.

### CLAIM FOR PRIORITY

Priority is hereby claimed to U.S. Provisional Patent Application Ser. No. 60/674,597 filed on Apr. 25, 2005.

### BACKGROUND

Delivering liquid or gel materials to an application surface using a tip is commonly performed in may fields. The prior art delivery tip is a solid piece. It may be pre-bent at the time of manufacturing to a desired angle.

### SUMMARY

A flexible delivery tip that can be bent by a clinical provider to any desired position is disclosed. The tip can be used to deliver all kinds of liquid or flowable materials and to guide fiber or wire.

### BRIEF DESCRIPTION OF DRAWINGS

FIG. 1a depicts a parts explosion drawing of an example flexible tip.

FIG. 1b depicts an example flexible tip.

FIG. 2a depicts a cross sectional view of a parts explosion drawing of an example flexible tip.

FIG. 2b depicts a cross sectional view of an example flexible tip.

FIG. 3 depicts a flocked tip.

FIG. 4 depicts a flexible tip delivering fiber or wire.

### DETAILED DESCRIPTION

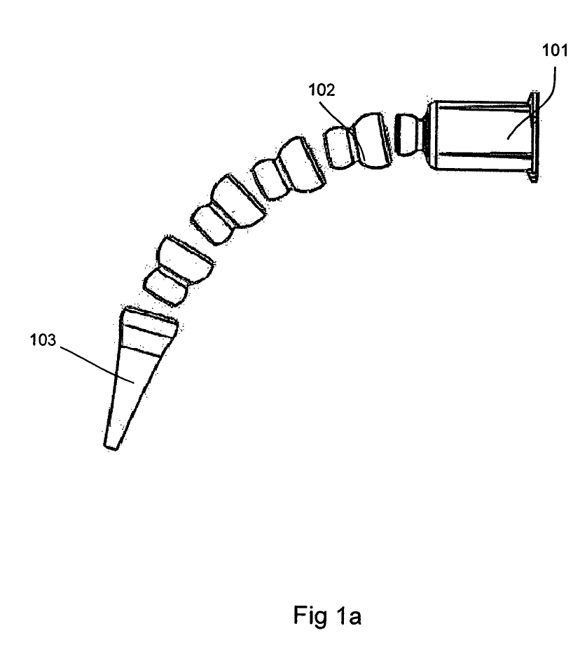

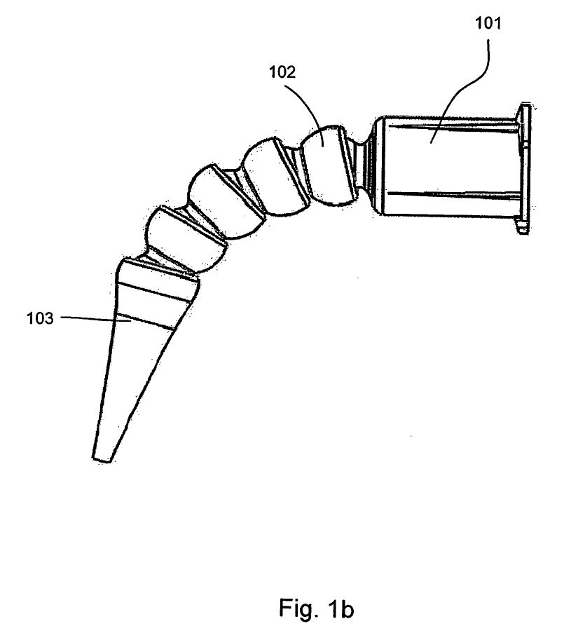

FIG. 1a depicts parts explosion drawing of an example flexible tip. The tip includes a connector 101 for connection to a syringe or other device. The connector 101 could include a standard luer lock or other attachment mechanism. A plurality of connection joints 102 are provided. They may be plastic and may snap together. Each connection joint is articulable with respect to the next connection joint that it is connected to. By articulating the connection joints, a health care provider may position an end or delivery piece 103 in a desired angular orientation. FIG. 1b depicts an example flexible delivery tip in assembled condition. Any desired number of connection joints may be used.

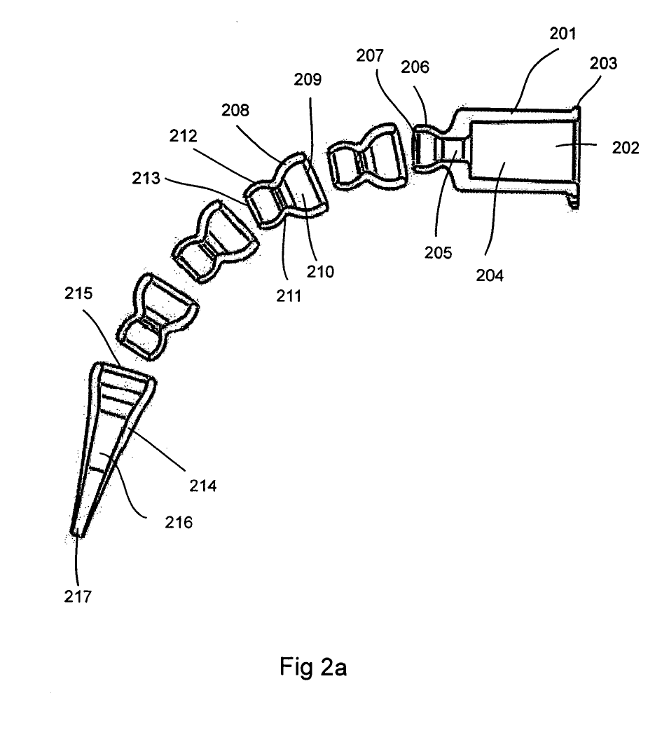

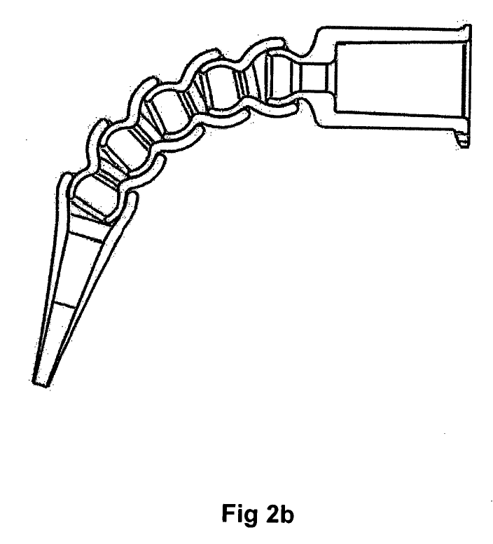

FIG. 2a depicts a cross-sectional view of a delivery tip. It includes a connector with a wall 201, an entry opening 202, a luer lock mechanical feature 203, an open cavity 204, a delivery channel 205 through which material may flow, a curved half-ball shape connection piece 206, and exit opening 207. The connection joint has a large hemispherical receptacle wall 208, an entry 209, a cavity 210, a neck with a channel 211, a small hemispherical protrusion wall 212, and exit 213. A delivery piece has a wall 214, an entry 215, a delivery channel 216, and an exit 217. FIG. 2b depicts the cross section of three major pieces connected together. The shape and size of connection joint is made in such that each piece can be connected to each other with tight fit. Thus, each piece will have the freedom to be articulated.

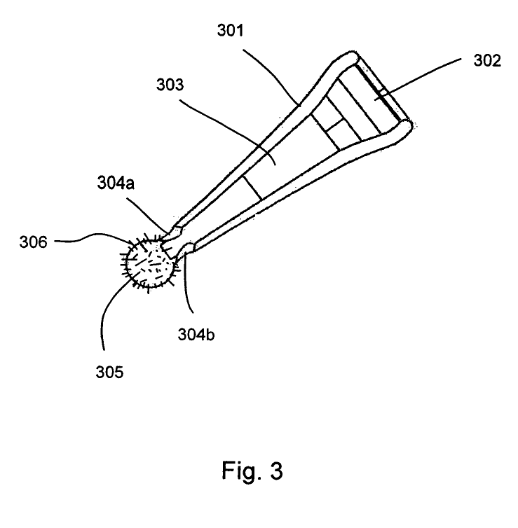

FIG. 3 depicts a flexible tip with a ball at the end and the ball is flocked with fiber. The flexible tip has an inner wall 301, an entry 302, a delivery channel 303, exit holes 304a and 304b, a ball 305, and flocked fiber 306 around the ball. Such deliver piece can be used with a connection joint and entry piece described above to construct a delivery tip. With a ball flocked with fiber at deliver end, the user can scrub the surface when the materials being delivered, such as in dental applications.

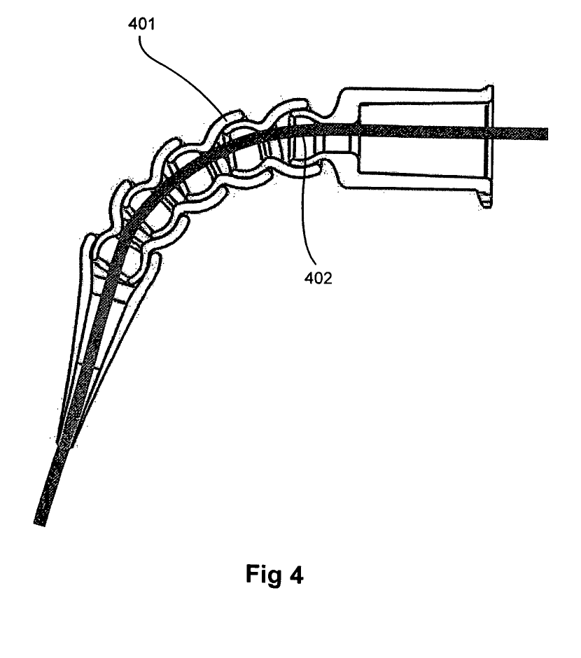

FIG. 4 depicts a cross section of a delivery device to guide a wire or optical fiber to a desire angle. The delivery device 401 uses articulable joints to guide a wire or optical fiber 402. By bending the tip, the wire or fiber can be formed at a desired angel.

While the present invention has been described and illustrated in conjunction with a number of specific embodiments, those skilled in the art will appreciate that variations and modifications may be made without departing from the principles of the inventions as herein illustrated, described and claimed. The present invention may be embodied in other specific forms without departing from their spirit or characteristics. The described embodiments are to be considered in all respects as only illustrative, and not restrictive. The scope of the invention is, therefore, indicated by the appended claims, rather than the foregoing description. All changes that come within the meaning and range of equivalency of the claims are to be embraced within their scope.

1. A delivery tip comprising:
an entry piece,
a connection piece, and an delivery piece;
wherein said pieces are articulable with respect to each other.

2. A device as recited in claim 1 further comprising a plurality of connection pieces.

3. A device as recited in claim 1 wherein any number of connection pieces may be snapped together to form a delivery tip of desired length.

4. A device as recited in claim 3 wherein said connection pieces are articulable to any desired angle.

5. A device as recited in claim 1 wherein said connection piece includes a hemispherical protrusion at one and a hemispherical receptacle at its other end, said protrusion being configured to snap into a receptacle on another piece, and said receptacle being configured to have a protrusion snapped into it.

6. A device as recited in claim 1 further comprising a channel through said tip.

7. A device as recited in claim 1 further comprising a ball flocked with fiber.

8. A device as recited in claim 1 wherein the device may be used to deliver flowable material, wire or fiber.
